(19) United States
(12) Patent Application Publication
(10) Pub. No.: US 2023/0277694 A1
(43) Pub. Date: Sep. 7, 2023

(54) OPHTHALMIC DYE COMPOSITION AND METHOD FOR ADMINISTERING SAME

(71) Applicant: Harrow IP, LLC, Nashville, TN (US)

(72) Inventors: Dennis Saadeh, Nashville, TN (US); William Wiley, Nashville, TN (US); Mark Baum, Nashville, TN (US)

(21) Appl. No.: 18/117,126

(22) Filed: Mar. 3, 2023

Related U.S. Application Data
(60) Provisional application No. 63/316,653, filed on Mar. 4, 2022.

(51) Int. Cl. A61K 49/00; A61K 9/00

(52) U.S. Cl. CPC A61K 49/006; A61K 9/0048; A61K 31/137

(57) ABSTRACT

The present disclosure encompasses methods and compositions for staining ocular structures in ophthalmologic procedures. The compositions generally include a mydriatic compound and a dye. The compositions may additionally include a pain-relieving compound and a second mydriatic compound. Also provided herein is a method of delivering a pharmaceutical composition, which includes administering to an eye of a subject an ophthalmic pharmaceutical composition of the present disclosure.

### CROSS-REFERENCE TO RELATED APPLICATIONS

This application claims priority to U.S. Provisional Application No. 63/316,653, filed Mar. 4, 2022, entitled “TRYPAN BLUE COMPOSITION AND METHOD FOR ADMINISTERING SAME”, the entire contents of which are incorporated by reference herein.

### FIELD OF THE DISCLOSURE

The present disclosure encompasses methods and compositions for staining ocular structures in ophthalmologic procedures. Accordingly, the present disclosure involves the fields of pharmacy, medicine, and chemistry.

### BACKGROUND

Ophthalmologic procedures, including cataract surgery and minimally invasive glaucoma surgery, involve placing stents, mesh, or other devices in or on different ocular structures. Accurate placement of these devices in relation to the ocular structures can be critical to the success of the procedure, and misplacement of the devices is common (Gillman, et al. 2019). In some cases, trypan blue is administered to the eye to aid ophthalmologists in identifying these ocular structures. However, the current process for using trypan blue requires injecting an air bubble into the eye before introducing the trypan blue, which impedes physiologic flow of the dye and can cause pain in the subject. Furthermore, high concentrations of trypan blue might cause too much staining, impeding the ophthalmologist’s view of ocular structures. One option is to instead dilate the pupils and thus expose the lens capsule. Visualization and exposure of the lens capsule can then aid practitioners in proper placement of intraocular devices. There is a need in the art, though, for a better visualization method that is easy to use.

### SUMMARY OF THE DISCLOSURE

Provided herein is an ophthalmic composition useful for dyeing a subject’s eye and inducing mydriasis. The composition includes at least one mydriatic compound, at least one pain-relieving compound, and a dye. The composition is operable to dilate a subject’s pupils and identify ocular structures, all while providing the subject pain relief. The composition may include a second mydriatic compound and a second pain relieving compound. The second mydriatic compound may be a non-steroidal anti-inflammatory drug.

In some embodiments, the first mydriatic compound includes epinephrine, phenylephrine, tropicamide, atropine, brimonidine, cyclopentolate, homatropine, 4-hydroxyamphetamine, or scopolamine, or pharmaceutically acceptable salts thereof. In some additional embodiments, the second mydriatic compound, when present, includes epinephrine, phenylephrine, tropicamide, atropine, brimonidine, cyclopentolate, homatropine, 4-hydroxyamphetamine, or scopolamine, or pharmaceutically acceptable salts thereof.

In some embodiments, the first mydriatic compound has a concentration in the composition from about 0.01% (w/v) to about 2% (w/v).

In some embodiments, the first pain-relieving compound includes lidocaine, proparacaine, tetracaine, dexamethasone, fluorometholone, fluocinolone, loteprednol, loteprednol, difluprednate, triamcinolone, prednisolone, medrysone, or rimexolone, or pharmaceutically acceptable salts thereof. In some additional embodiments, the second pain-relieving compound, when present, includes lidocaine, proparacaine, tetracaine, dexamethasone, fluorometholone, fluocinolone, loteprednol, loteprednol, difluprednate, triamcinolone, prednisolone, medrysone, or rimexolone, or pharmaceutically acceptable salts thereof.

In some embodiments, the first pain-relieving compound has a concentration from about 0.5% (w/v) to about 2% (w/v).

In some embodiments, the dye includes trypan blue, fluorescein, lissamine green, rose Bengal, indocyanine green, triamcinolone acetonide, bromophenol blue, patent blue, brilliant blue G (acid blue), or a combination thereof. In some additional embodiments, the dye has a concentration from about 0.01% (w/v) to about 0.25% (w/v).

In preferred embodiments, the composition is essentially free of preservatives and sulfites.

Also described herein is a method for administering a composition useful for dyeing a subject’s eye and inducing mydriasis. The method includes administering a composition to a subject’s eye, wherein the composition comprises at least one mydriatic compound, at least one pain-relieving compound, and trypan blue. The composition may be administered intracamerally via a cannula, or it may be administered topically. The method may further include using a video camera, a computer, and a display monitor to aid an ophthalmologist in identifying ocular structures.

### DETAILED DESCRIPTION

It is to be understood that this disclosure is not limited to the particular methods, compositions, or materials specified herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL” The endpoint may also be based on the variability allowed by an appropriate regulatory body, such as the FDA, USP, etc.

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition’s nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein, “subject” refers to a vertebrate, preferably, a mammal. Examples of subjects include humans, and may also include other animals such as horses, pigs, cattle, dogs, cats, rabbits, and aquatic mammals. In one aspect, the subject can be a human.

As used herein, “pain-relieving compound” refers to any drug, agent, formulation, compound, pharmaceutical, or other composition that relieves pain experienced by a subject. Some examples of pain-relieving compounds include non-steroidal anti-inflammatory drugs, steroidal anti-inflammatory drugs, opioids, acetaminophen, analgesics, anesthetics, and other pain-relieving compounds known in the art.

As used herein, “mydriatic compound” refers to any drug, agent, formulation, compound, pharmaceutical, or other composition that induces the dilation of a subject’s pupil. Some examples of mydriatic compounds include stimulants, anticholinergics, serotonergics, dissociatives, GABAergics, adrenergic agonists, and other mydriatic compounds known in the art.

As used herein, “ocular structure” may refer to anatomical features of a subject’s eye, such as the lens, cornea, iris, pupil, anterior chamber, posterior chamber, uvea, ciliary body, choroid, or other anatomical features of the eye recognized by those skilled in the art. Additionally, “ocular structure” may also refer to medical devices placed in or on the eye, such as mesh, stents, or other devices, as well as incisions made in the eye.

### I. Compositions

Described herein are ophthalmic compositions comprising a first mydriatic compound, a first pain-relieving compound, and a dye. In some embodiments, the composition may further comprise a second mydriatic compound and second pain-relieving compound. In some aspects, the second pain-relieving compound is a non-steroidal anti-inflammatory agent. The composition is operable to dilate a subject’s pupils, reveal critical ocular structures, and provide pain relief to the subject.

### (A) Mydriatic Compound

A composition of the present disclosure comprises at least one mydriatic compound. Mydriatic compounds, and methods of making or procuring mydriatic compounds, are known in the art. The first mydriatic compound may include epinephrine, phenylephrine, tropicamide, atropine, brimonidine, cyclopentolate, homatropine, 4-hydroxyamphetamine, scopolamine, or another mydriatic compound or pharmaceutically acceptable salt thereof.

In some embodiments, the first mydriatic compound may have a concentration from about 0.01% (w/v) to about 2% (w/v). In some examples, the first mydriatic compound may have a concentration of about 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), 1.0% (w/v), 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), or about 2.0% (w/v). In some examples, the first mydriatic compound may have a concentration from about 0.01% (w/v) to about 0.02% (w/v), about 0.02% (w/v) to about 0.03% (w/v), about 0.03% (w/v) to about 0.04% (w/v), about 0.04% (w/v) to about 0.05% (w/v), about 0.05% (w/v) to about 0.06% (w/v), about 0.06% (w/v) to about 0.07% (w/v), about 0.07% (w/v) to about 0.08% (w/v), about 0.08% (w/v) to about 0.09% (w/v), about 0.09% (w/v) to about 0.1% (w/v), about 0.1% (w/v) to about 0.2% (w/v), about 0.2% (w/v) to about 0.3% (w/v), about 0.3% (w/v) to about 0.4% (w/v), about 0.4% (w/v) to about 0.5% (w/v), about 0.5% (w/v) to about 0.6% (w/v), about 0.6% (w/v) to about 0.7% (w/v), about 0.7% (w/v) to about 0.8% (w/v), about 0.8% (w/v) to about 0.9% (w/v), about 0.9% (w/v) to about 1% (w/v), about 1% (w/v) to about 1.1% (w/v), about 1.1% (w/v) to about 1.2% (w/v), about 1.2% (w/v) to about 1.3% (w/v), about 1.3% (w/v) to about 1.4% (w/v), about 1.4% (w/v) to about 1.5% (w/v), about 1.5% (w/v) to about 1.6% (w/v), about 1.6% (w/v) to about 1.7% (w/v), about 1.7% (w/v) to about 1.8% (w/v), about 1.8% (w/v) to about 1.9% (w/v), about 1.9% (w/v) to about 2% (w/v), about 0.01% (w/v) to about 0.05% (w/v), about 0.01% (w/v) to about 0.1% (w/v), about 0.01% (w/v) to about 0.5% (w/v), about 0.01% (w/v) to about 1% (w/v), about 0.01% (w/v) to about 2% (w/v), about 0.05% (w/v) to about 2% (w/v), about 0.1% (w/v) to about 2% (w/v), about 0.5% (w/v) to about 2% (w/v), or about 1% (w/v) to about 2% (w/v).

In some embodiments, the first mydriatic compound may be epinephrine or a pharmaceutically acceptable salt thereof at a concentration from about 0.01% (w/v) to about 0.05% (w/v). Thus, the concentration of epinephrine may be about 0.01% (w/v), 0.02% (w/v), 0.03% (w/v), 0.04% (w/v), or about 0.05% (w/v). In some examples, the concentration of epinephrine may be from about 0.01% (w/v) to about 0.02% (w/v), about 0.02% (w/v) to about 0.03% (w/v), about 0.03% (w/v) to about 0.04% (w/v), or about 0.04% (w/v) to about 0.05% (w/v). In one example, the concentration of epinephrine is 0.025% (w/v).

In other embodiments, the first mydriatic compound may be phenylephrine at a concentration from about 0.01% (w/v) to about 2% (w/v). Thus, the concentration of phenylephrine may be about 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), 1% (w/v), 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), or about 2% (w/v). In some examples, the phenylephrine may have a concentration from about 0.01% (w/v) to about 0.02% (w/v), about 0.02% (w/v) to about 0.03% (w/v), about 0.03% (w/v) to about 0.04% (w/v), about 0.04% (w/v) to about 0.05% (w/v), about 0.05% (w/v) to about 0.06% (w/v), about 0.06% (w/v) to about 0.07% (w/v), about 0.07% (w/v) to about 0.08% (w/v), about 0.08% (w/v) to about 0.09% (w/v), about 0.09% (w/v) to about 0.1% (w/v), about 0.1% (w/v) to about 0.2% (w/v), about 0.2% (w/v) to about 0.3% (w/v), about 0.3% (w/v) to about 0.4% (w/v), about 0.4% (w/v) to about 0.5% (w/v), about 0.5% (w/v) to about 0.6% (w/v), about 0.6% (w/v) to about 0.7% (w/v), about 0.7% (w/v) to about 0.8% (w/v), about 0.8% (w/v) to about 0.9% (w/v), about 0.9% (w/v) to about 1% (w/v), about 1% (w/v) to about 1.1% (w/v), about 1.1% (w/v) to about 1.2% (w/v), about 1.2% (w/v) to about 1.3% (w/v), about 1.3% (w/v) to about 1.4% (w/v), about 1.4% (w/v) to about 1.5% (w/v), about 1.5% (w/v) to about 1.6% (w/v), about 1.6% (w/v) to about 1.7% (w/v), about 1.7% (w/v) to about 1.8% (w/v), about 1.8% (w/v) to about 1.9% (w/v), or about 1.9% (w/v) to about 2% (w/v). In one example, the concentration of phenylephrine is 1.5% (w/v).

In yet other embodiments, the first mydriatic compound may be tropicamide with a concentration from about 0.1% (w/v) to about 0.5% (w/v). Thus, the concentration of tropicamide may be from about 0.1% (w/v), 0.2% (w/v), 0.3% (w/v), 0.4% (w/v), or about 0.5% (w/v). In some examples, the concentration of tropicamide may be about 0.1% (w/v) to about 0.2% (w/v), about 0.2% (w/v) to about 0.3% (w/v), about 0.3% (w/v) to about 0.4% (w/v), or about 0.4% (w/v) to about 0.5% (w/v). In one example, the concentration of tropicamide is 0.3% (w/v).

In yet other embodiments, the composition may further include a second mydriatic compound. The second mydriatic compound may be any one of the mydriatic compounds described above in the same ranges of concentrations and are thus incorporated herein by reference.

### (B) Pain-Relieving Compound

A composition of the present disclosure comprises at least one pain-relieving compound. Pain-relieving compounds, as well as methods of making and procuring pain-relieving compounds, are known in the art. In some examples, the first pain-relieving compound may include lidocaine, proparacaine, tetracaine, dexamethasone, fluorometholone, fluocinolone, loteprednol, loteprednol, difluprednate, triamcinolone, prednisolone, medrysone, rimexolone, or any other pain-relieving compound or pharmaceutically acceptable salts thereof.

The composition includes a first pain-relieving compound. In some embodiments, the pain-relieving compound may have a concentration from about 0.5% (w/v) to about 2% (w/v). Therefore, the first pain-relieving compound may have a concentration of about 0.5% (w/v), 0.6% (w/v), 0.7% (w/v), 0.8% (w/v), 0.9% (w/v), 1% (w/v), 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), or about 2% (w/v). In some examples, the concentration of the first pain-relieving compound may be about 0.5% (w/v) to about 0.6% (w/v), 0.6% (w/v) to about 0.7% (w/v), about 0.7% (w/v) to about 0.8% (w/v), about 0.8% (w/v) to about 0.9% (w/v), about 0.9% (w/v) to about 1% (w/v), about 1% (w/v) to about 1.1% (w/v), about 1.1% (w/v) to about 1.2% (w/v), about 1.2% (w/v) to about 1.3% (w/v), about 1.3% (w/v) to about 1.4% (w/v), about 1.4% (w/v) to about 1.5% (w/v), about 1.5% (w/v) to about 1.6% (w/v), about 1.6% (w/v) to about 1.7% (w/v), about 1.7% (w/v) to about 1.8% (w/v), about 1.8% (w/v) to about 1.9% (w/v), about 1.9% (w/v) to about 2% (w/v), about 0.5% (w/v) to about 1% (w/v), about 0.5% (w/v) to about 1.5% (w/v), about 0.5% (w/v) to about 2% (w/v), about 1% (w/v) to about 2% (w/v), or about 1.5% (w/v) to about 2% (w/v). In some aspects, the first pain-relieving compound does not include a non-steroidal anti-inflammatory agent.

In some embodiments, the first pain-relieving compound includes lidocaine or a pharmaceutically acceptable salt thereof in a concentration from about 0.5% (w/v) to about 2% (w/v). Thus, the concentration of lidocaine may be about 0.5% (w/v), 0.6% (w/v), 0.7% (w/v), 0.8% (w/v), 0.9% (w/v), 1% (w/v), 1.1% (w/v), 1.2% (w/v), 1.3% (w/v), 1.4% (w/v), 1.5% (w/v), 1.6% (w/v), 1.7% (w/v), 1.8% (w/v), 1.9% (w/v), or about 2% (w/v). In some embodiments, the first pain-relieving compound is lidocaine. In some examples, the concentration of lidocaine may be about 0.5% (w/v) to about 0.6% (w/v), about 0.6% (w/v) to about 0.7% (w/v), about 0.7% (w/v) to about 0.8% (w/v), about 0.8% (w/v) to about 0.9% (w/v), about 0.9% (w/v) to about 1% (w/v), about 1% (w/v) to about 1.1% (w/v), about 1.1% (w/v) to about 1.2% (w/v), about 1.2% (w/v) to about 1.3% (w/v), about 1.3% (w/v) to about 1.4% (w/v), about 1.4% (w/v) to about 1.5% (w/v), about 1.5% (w/v) to about 1.6% (w/v), about 1.6% (w/v) to about 1.7% (w/v), about 1.7% (w/v) to about 1.8% (w/v), about 1.8% (w/v) to about 1.9% (w/v), or about 1.9% (w/v) to about 2% (w/v). In some examples, the concentration of lidocaine is about 1% (w/v).

In some embodiments, the composition includes a second pain-relieving compound. The second pain-relieving compound may be a non-steroidal anti-inflammatory drug, a corticosteroid, a local anesthetic, or a cycloplegic. In some embodiments, the second pain-relieving compound is a non-steroidal anti-inflammatory drug. In some embodiments, the non-steroidal anti-inflammatory drug may be diclofenac, ketorolac, bromfenac, etodolac, sulindac, aceclofenac, nepafenac, tolmetin, indomethacin, nabumetone, ketoprofen, dexketoprofen, ibuprofen, flurbiprofen, dexibuprofen, fenoprofen, loxoprofen, oxaprozin, naproxen, aspirin, salicylic acid, diflunisal, salsalate, mefenamic acid, meclofenamic acid, flufenamic acid, tolfenamic acid, meloxicam, piroxicam, ternoxicam, droxicam, lornoxicam, isoxicam, celecoxib, rofecoxib, valdecoxib, parecoxib, lumiracoxib, etoricoxib, firocoxib, nimesulide, clonixin, licofelone, or any other non-steroidal anti-inflammatory drug or pharamaceutically acceptable salts thereof. The concentration of the non-steroidal anti-inflammatory drug may be from about 0.01% (w/v) to 0.5% (w/v). Thus, the concentration of the non-steroidal anti-inflammatory drug may be about 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.2% (w/v), 0.3% (w/v), 0.4% (w/v), or about 0.5% (w/v). In some examples, the concentration of the non-steroidal anti-inflammatory drug may be from about 0.01% (w/v) to about 0.02% (w/v), about 0.02% (w/v) to about 0.03% (w/v), about 0.03% (w/v) to about 0.04% (w/v), about 0.04% (w/v) to about 0.05% (w/v), about 0.05% (w/v) to about 0.06% (w/v), about 0.06% (w/v) to about 0.07% (w/v), about 0.07% (w/v) to about 0.08% (w/v), about 0.08% (w/v) to about 0.09% (w/v), about 0.09% (w/v) to about 0.1% (w/v), about 0.1% (w/v) to about 0.2% (w/v), about 0.2% (w/v) to about 0.3% (w/v), about 0.3% (w/v) to about 0.4% (w/v), or about 0.4% (w/v) to about 0.5% (w/v). In some examples, the second pain-relieving compound is diclofenac at a concentration from about 0.01% (w/v) to about 0.5% (w/v). In other examples, the concentration of diclofenac is 0.1% (w/v).

### (C) Dye

The composition of the present disclosure comprises a dye. Dyes for ophthalmic use, and methods of making or procuring dyes for ophthalmic use, are known in the art. In some examples, the dye may include trypan blue, fluorescein, lissamine green, rose Bengal, indocyanine green, triamcinolone acetonide, bromophenol blue, patent blue, brilliant blue G (acid blue), or any other dye for ophthalmic use and combinations thereof.

The concentration of the dye in the composition may be from about 0.01% (w/v) to about 0.25% (w/v). Thus, the concentration of the dye may be about 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), 0.2% (w/v), 0.21% (w/v), 0.22% (w/v), 0.23% (w/v), 0.24% (w/v), or about 0.25% (w/v). In some examples, the concentration of the dye may be from about 0.01% (w/v) to about 0.05% (w/v), about 0.01% (w/v) to about 0.1% (w/v), about 0.01% (w/v) to about 0.15% (w/v), about 0.01% (w/v) to about 0.2% (w/v), about 0.01% (w/v) to about 0.25% (w/v), about 0.05% (w/v) to about 0.25% (w/v), about 0.1% (w/v) to about 0.25% (w/v), about 0.15% (w/v) to about 0.25% (w/v), or about 0.2% (w/v) to about 0.25% (w/v).

In some embodiments, the composition includes trypan blue. The concentration of trypan blue in the composition may be from about 0.01% (w/v) to about 0.25% (w/v). Thus, the concentration of trypan blue may be about 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.11% (w/v), 0.12% (w/v), 0.13% (w/v), 0.14% (w/v), 0.15% (w/v), 0.16% (w/v), 0.17% (w/v), 0.18% (w/v), 0.19% (w/v), 0.2% (w/v), 0.21% (w/v), 0.22% (w/v), 0.23% (w/v), 0.24% (w/v), or about 0.25% (w/v). In some examples, the concentration of trypan blue is from about 0.01% (w/v) to about 0.02% (w/v), about 0.02% (w/v) to about 0.03% (w/v), about 0.03% (w/v) to about 0.04% (w/v), about 0.04% (w/v) to about 0.05% (w/v), about 0.05% (w/v) to about 0.06% (w/v), about 0.06% (w/v) to about 0.07% (w/v), about 0.07% (w/v) to about 0.08% (w/v), about 0.08% (w/v) to about 0.09% (w/v), about 0.09% (w/v) to about 0.1% (w/v), about 0.1% (w/v) to about 0.11% (w/v), about 0.11% (w/v) to about 0.12% (w/v), about 0.12% (w/v) to about 0.13% (w/v), about 0.13% (w/v) to about 0.14% (w/v), about 0.14% (w/v) to about 0.15% (w/v), about 0.15% (w/v) to about 0.16% (w/v), about 0.16% (w/v) to about 0.17% (w/v), about 0.17% (w/v) to about 0.18% (w/v), about 0.18% (w/v) to about 0.19% (w/v), about 0.19% (w/v) to about 0.2% (w/v), about 0.2% (w/v) to about 0.21% (w/v), about 0.21% (w/v) to about 0.22% (w/v), about 0.22% (w/v) to about 0.23% (w/v), about 0.23% (w/v) to about 0.24% (w/v), or about 0.24% (w/v) to about 0.25% (w/v). In some examples, the concentration of trypan blue in the composition is from about 0.01% (w/v) to about 0.15% (w/v). In still other examples, the concentration of trypan blue is 0.05% (w/v).

### (D) Exemplary Embodiments

A list of exemplary embodiments of the disclosure is presented in Table 1.

| | | | | |
|---|---|---|---|---|
| | Embodiment 1 | Embodiment 2 | Embodiment 3 | Embodiment 4 |
| First Mydriatic Compound | Epinephrine (0.025% (w/v)) | Phenylephrine (1.5% (w/v)) | Tropicamide (0.3% (w/v)) | Tropicamide (0.3% (w/v)) |
| Seccond Mydriatic Compound | N/A | N/A | N/A | Phenylephrine (1.5% (w/v)) |
| First Pain-Relieving Compound | Lidocaine (1% (w/v)) | Lidocaine (1% (w/v)) | Lidocaine (1% (w/v)) | Lidocaine (1% (w/v)) |
| Second Pain-Relieving Compound | N/A | N/A | N/A | Diclofenac (0.1% (w/v)) |
| Dye | Trypan blue (0.05% (w/v)) | Trypan blue (0.05% (w/v)) | Trypan blue (0.05% (w/v)) | Trypan blue (0.05% (w/v)) |

### (E) Pharmaceutical Formulations

In some embodiments, compositions of the present disclosure are pharmaceutically-acceptable formulations. The pharmaceutically-acceptable formulations may include pharmaceutically-acceptable excipients, including solvents, pH adjusting agents, buffering agents, antioxidants, tonicity modifying agents, osmotic adjusting agents, chelating agents, preservatives, antibacterial agents, stabilizing agents, viscosity adjusting agents, surfactants, or any other pharmaceutically-acceptable excipients known in the art or combinations thereof. Accordingly, the pharmaceutical composition of the disclosure may include pharmaceutically-acceptable excipients such as monosodium phosphate, disodium phosphate, sodium chloride, EDTA disodium, potassium chloride, calcium chloride, sodium acetate, sodium citrate, sodium hydroxide, dextrose anhydrous, sodium bicarbonate, acetylcysteine, boric acid, citric acid, glycerin, monopotassium phosphate, dipotassium phosphate, hypromellose, polyethylene glycol 300, polyethylene glycol 400, carboxymethyl cellulose, hydroxyethyl cellulose, methylcellulose, dextran 70, polysorbate 80, propylene glycol, gelatin, polyvinyl alcohol, povidone, or other pharmaceutically-acceptable excipients. In further embodiments, the composition is essentially free of preservatives and sulfites (i.e., less than 0.001% (w/v)).

The pharmaceutically-acceptable excipient may include a chelation agent. Chelation agents are generally known in the art, and include EDTA salts (e.g., EDTA disodium, EDTA trisodium) and DTPA. The chelation agent may have a concentration in the formulation from about 0.01% (w/v) to about 1% (w/v), such as from about 0.01% (w/v) to about 0.05% (w/v), about 0.01% (w/v) to about 0.1% (w/v), about 0.01% (w/v) to about 0.5% (w/v), about 0.01% (w/v) to about 1% (w/v), about 0.05% (w/v) to about 1% (w/v), or about 0.1% (w/v) to about 1% (w/v). Further, the chelation agent may have a concentration in the formulation of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), or about 1% (w/v). Preferably, the chelation agent comprises EDTA disodium.

The pharmaceutically-acceptable excipient may include a tonicity modifying agent. Tonicity modifying agent are generally known in the art and may include chloride salts (e.g., potassium chloride, sodium chloride, calcium chloride), dextrose, glycerin, mannitol, boric acid, or combinations thereof. The tonicity modifying agent may have a concentration in the formulation from about 0.01% (w/v) to about 1% (w/v), such as from about 0.01% (w/v) to about 0.05% (w/v), about 0.01% (w/v) to about 0.1% (w/v), about 0.01% (w/v) to about 0.5% (w/v), about 0.01% (w/v) to about 1% (w/v), about 0.05% (w/v) to about 1% (w/v), or about 0.1% (w/v) to about 1% (w/v). Further, the tonicity modifying agent may have a concentration in the formulation of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), or about 1% (w/v).

The pharmaceutically-acceptable excipient may include a buffering agent. Buffering agents are generally known in the art and may include sodium bicarbonate, boric acid, sodium citrate, sodium acetate, or other buffering agents known in the art and combinations thereof. The buffering agent may have a concentration in the formulation from about 0.01% (w/v) to about 1% (w/v), such as from about 0.01% (w/v) to about 0.05% (w/v), about 0.01% (w/v) to about 0.1% (w/v), about 0.01% (w/v) to about 0.5% (w/v), about 0.01% (w/v) to about 1% (w/v), about 0.05% (w/v) to about 1% (w/v), or about 0.1% (w/v) to about 1% (w/v). Further, the buffering agent may have a concentration in the formulation of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), or about 1% (w/v).

The pharmaceutically-acceptable excipient may include a stabilizing agent. Stabilizing agents are generally known in the art and may include acetylcysteine. The stabilizing agent may have a concentration in the formulation from about 0.01% (w/v) to about 1% (w/v), such as from about 0.01% (w/v) to about 0.05% (w/v), about 0.01% (w/v) to about 0.1% (w/v), about 0.01% (w/v) to about 0.5% (w/v), about 0.01% (w/v) to about 1% (w/v), about 0.05% (w/v) to about 1% (w/v), or about 0.1% (w/v) to about 1% (w/v). Further, the stabilizing agent may have a concentration in the formulation of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), or about 1% (w/v).

The pharmaceutically-acceptable excipient may include a surfactant. Surfactants are generally known in the art and may include polysorbates (e.g., polysorbate 80), poloxamers, or other surfactants known in the art and combinations thereof. The stabilizing agent may have a concentration in the formulation from about 0.01% (w/v) to about 1% (w/v), such as from about 0.01% (w/v) to about 0.05% (w/v), about 0.01% (w/v) to about 0.1% (w/v), about 0.01% (w/v) to about 0.5% (w/v), about 0.01% (w/v) to about 1% (w/v), about 0.05% (w/v) to about 1% (w/v), or about 0.1% (w/v) to about 1% (w/v). Further, the stabilizing agent may have a concentration in the formulation of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), or about 1% (w/v).

The pharmaceutically-acceptable formulations may also include a pharmaceutically-acceptable carrier. A pharmaceutically-acceptable carrier may be any substance that serves as a vehicle for improving the efficiency of delivery and the effectiveness of the pharmaceutical composition. In some examples of the present disclosure, the pharmaceutically-acceptable carrier may be water (e.g., deionized sterile water or water sterile for injection).

In some embodiments, the formulation may be a solution or a suspension.

The pharmaceutical formulation may have a pH from about 4.0 to about 8.0, such as from about 5.0 to about 7.0. In some aspects, the pharmaceutical formulation may have a pH from about 4.0 to about 5.0, about 4.0 to about 6.0, about 4.0 to about 7.0, about 4.0 to about 8.0, about 5.0 to about 5.5, about 5.0 to about 6.0, about 5.0 to about 6.5, about 5.0 to about 7.0, about 5.0 to about 8.0, about 6.0 to about 6.5, about 6.0 to about 7.0, about 6.0 to about 8.0, or about 7.0 to about 8.0. In still further aspects, the pharmaceutical formulation may have a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or about 8.0.

### II. Methods

Described herein are methods of delivering a composition described in Section I above. In some embodiments, the method comprises administering to the eye of a subject a therapeutically effective amount of a composition comprising a first mydriatic compound, a first pain-relieving compound, and trypan blue. As used herein, the term “therapeutically effective amount” means an amount that leads to measurable and beneficial effects for the subject administered the substance. In some embodiments, a method of the present disclosure may result in dilation of the subject’s eyes, injection of an amount of dye sufficient to allow an ophthalmologist to identify ocular structures, and reduction in the amount of pain experienced by the subject.

In some embodiments, the method may further comprise administering the composition via a cannula. In such embodiments, a cannula is used to inject the formulation intracamerally into the subject’s eye. In some examples, the volume of the composition injected may be from about 0.3 mL to about 0.7 mL, such as about 0.3 mL, 0.4 mL, 0.5 mL, 0.6 mL, or about 0.7 mL.

In some embodiments, the method may further comprise administering the formulation topically. In such embodiments, the formulation may be administered via eyedrops into the subject’s eye. In one example, two drops are administered into the subject’s eye, followed by another two drops 15 minutes after the first two drops were administered.

In some embodiments, the method may further comprise identifying ocular structures by using a camera, a computer, and a display screen, wherein the camera is operable to record a video of the subject’s eye, the computer is operable to alter at least a portion of the video, and the display screen is operable to display the altered video. In some embodiments, the portion of the video that is altered includes the areas where the composition of Section I has been applied and has dyed ocular structures. The alteration may include changing the color of the dyed ocular structures, forming an outline around the dyed ocular structures, inserting text over the dyed ocular structures, inserting a shape over the dyed ocular structures, or other alterations. The alteration would assist an ophthalmologist in identifying ocular structures and may also identify areas where devices should be placed or where incisions should be made.

Further provided herein is a method of making a composition of the present disclosure. First, water is heated to a temperature from about 50° C. to about 70° C., such as about 50° C., 55° C., 60° C., 65° C., or about 70° C. In particular emboidments, the water is heated to a temperature from about 55° C. to about 60° C.

After the water is heated, the first mydriatic compound is added to the water and mixed until dissolved. Once the first mydriatic compound is dissolved, the solution is allowed to cool to room temperature (i.e., about 20° C. to about 25° C.).

When the solution has cooled to room temperature, a second mydriatic compound, a one or more pharmaceutically-acceptable excipients, a second mydriatic compound, a pain-relieving compound, and/or a dye may be added to the solution and mixed until dissolved. These may be added in any order desired or in any order to achieve full dissolution of all components. In a particular embodiment, a pharmaceutically-acceptable excipient is added first, optionally followed by a second mydriatic compound, optionally followed by a first pain-relieving compound, optionally followed by one or more additional pharmaceutically-acceptable excipients, optionally followed by a second pain-relieving compound, followed by a dye.

Next, the pH of the solution may be adjusted using a pH adjusting agent, such as HCl or NaOH. The pH of the solution may be adjusted to a pH from about 4.0 to about 8.0, such as from about 5.0 to about 7.0.

Once the desired pH has been reached, additional water may be added to bring the solution to a final volume.

In one example, a solution made using the following method. First, a de-pyrogenated mixing vessel is filled with sterile water for injection to a predetermined amount. The vessel with the sterile water for injection is then heated on a heating place to about 60° C. Once the sterile water for injection is heated to about 55-60° C., a first mydriatic compound is added to the vessel. The solution is mixed until the first mydriatic compound is dissolved. Once the first mydriatic compound is dissolved, the solution is cooled to room temperature. Once the solution has cooled, excipients may be added. After adding each excipient, the formulation is mixed until the excipient is dissolved before adding another excipient. After the excipients are added, a second mydriatic compound may optionally be added. After the excipients are added, or after the second mydriatic compound is added, a first pain-relieving compound may be added. Once the first pain-relieving compound is dissolved, one or more second excipients may be added. After the one or more second excipients have dissolved, a second pain-relieving compound may optionally be added. After all the previous components have dissolved, the trypan blue may be added. Once all components are dissolved in the solution, the pH of the solution may be adjusted to a predetermined target pH by adding dropwise an acid or a base. Once the pH of the solution reaches the target value, more sterile water for injection may be added to bring the solution to a final predetermined volume. The solution may then be filtered into an appropriate sterile dispensing container.

### EXAMPLES

### Example 1

A pharmaceutically-acceptable formulation was prepared as described below. The following products were used in the amounts and concentrations specified:
(a) about 0.300 g of phenylephrine hydrochloride;
(b) about 0.020 g of tropicamide;
(c) about 1.000 g of lidocaine;
(d) about 0.010 g of diclofenac sodium;
(e) about 0.100 g of edetate disodium
(f) about 0.352 g of powdered boric acid;
(g) about 0.386 g of sodium chloride;
(h) about 0.05 g of trypan blue;
(i) about 0.100 mL of polysorbate 80;
(j) about 100.0 mL of water sterile for injection; and
(k) a small quantity of 20% aqueous solution of sodium hydroxide (for adjusting pH).

To a calibrated beaker containing about 80.0 mL of sterile water for injection heated to 55-60° C., the tropicamide was added. The tropicamide was added slowly to avoid clumping and was mixed at 3000 ± 500 rpm. After the tropicamide is fully dissolved (30-40 minutes), the heat was turned off and the solution was allowed to cool to room temperature.

Once the solution reached room temperature, the following ingredients were added in the following order and mixed until dissolved in the solution: sodium chloride, edetate disodium, boric acid, phenylephrine, lidocaine, polysorbate 80, and diclofenac. It was observed that if the diclofenac was added before polysorbate 80, the diclofenac would not dissolve. After the diclofenac was added, the solution was mixed for 30 minutes. Once the solution was mixed, the trypan blue was added and the solution was mixed.

Once the solution was mixed and all components had dissolved, the pH of the solution was measured and adjusted to a pH of 6.5 ± 0.1 by adding the sodium hydroxide dropwise. The solution was then filtered using a 0.22-micron filter into a sterile dispensing container.

### Example 2

A pharmaceutically-acceptable formulation was prepared as described below. The following products were used in the amounts and concentrations specified:
(a) about 0.030 g of epinephrine hydrochloride;
(b) about 0.750 g of lidocaine hydrochloride;
(c) about 0.100 g of edetate disodium;
(d) about 0.020 g of potassium chloride;
(e) about 0.400 g of sodium chloride;
(f) about 0.010 g of calcium chloride;
(g) about 0.160 g of sodium acetate;
(h) about 0.050 g of sodium citrate;
(i) about 0.040 g of dextrose anhydrous;
(j) about 0.100 g of sodium bicarbonate;
(k) about 0.100 g of acetylcysteine;
(l) about 0.050 g of trypan blue;
(m)about 100 mL of water for injection; and
(n) a small quantity of 10% aqueous solution of sodium hydroxide or hydrochloric acid (for adjusting pH).

To a calibrated beaker containing about 80.0 mL of sterile water for injection, the following ingredients were added in the following order and mixed until dissolved in the solution: epinephrine, lidocaine, edetate disodium, potassium chloride, sodium chloride, calcium chloride, sodium acetate, sodium citrate, dextrose, sodium bicarbonate, acetylcysteine, and trypan blue.

Once the solution was mixed and all components had dissolved, the pH of the solution was measured and adjusted to a pH of 5.25 ± 0.1 by adding the sodium hydroxide dropwise. The solution was then filtered using a 0.22-micron filter into a sterile dispensing container.

### Example 3

A pharmaceutically-acceptable formulation was prepared as described below. The following products were used in the amounts and concentrations specified:
(a) about 1.500 g of phenylephrine hydrochloride;
(b) about 1.000 g of lidocaine hydrochloride;
(c) about 0.100 g of edetate disodium;
(d) about 0.352 g of boric acid;
(e) about 0.050 g of trypan blue;
(f) about 100 mL of water for injection; and
(g) a small quantity of 10% aqueous solution of sodium hydroxide or hydrochloric acid (for adjusting pH).

To a calibrated beaker containing about 80.0 mL of sterile water for injection, the following ingredients were added in the following order and mixed until dissolved in solution: phenylephrine, lidocaine, edetate disodium, boric acid, and trypan blue.

Once the solution was mixed and all components had dissolved, the pH of the solution was measured and adjusted to a pH of 6.5 ± 0.1 by adding the sodium hydroxide or hydrochloric acid dropwise. The solution was then filtered using a 0.22-micron filter into a sterile dispensing container.

What is claimed is:

1. An ophthalmic pharmaceutical composition, the composition comprising:
a first mydriatic compound,
a first pain-relieving compound,
and a dye.

2. The composition of claim 1, wherein the first mydriatic compound comprises epinephrine, phenylephrine, tropicamide, atropine, brimonidine, cyclopentolate, homatropine, 4-hydroxyamphetamine, or scopolamine, or pharmaceutically acceptable salts thereof.

3. The composition of claim 1 wherein the first mydriatic compound has a concentration from about 0.01% (w/v) to about 2% (w/v).

4. The composition of claim 1, wherein the first pain-relieving compound comprises lidocaine, proparacaine, tetracaine, dexamethasone, fluorometholone, fluocinolone, loteprednol, loteprednol, difluprednate, triamcinolone, prednisolone, medrysone, or rimexolone, or pharmaceutically acceptable salts thereof.

5. The composition of claim 1, wherein the dye comprises trypan blue, fluorescein, lissamine green, rose Bengal, indocyanine green, triamcinolone acetonide, bromophenol blue, patent blue, brilliant blue G (acid blue), or a combination thereof.

6. The composition of claim 1 wherein the first pain-relieving compound has a concentration from about 0.5% (w/v) to about 2% (w/v).

7. The composition of claim 1 wherein the first mydriatic compound comprises epinephrine and the first pain-relieving compound comprises lidocaine.

8. The composition of claim 7 wherein the epinephrine has a concentration from about 0.01% (w/v) to about 0.05% (w/v).

9. The composition of claim 7 wherein the lidocaine has a concentration from about 0.5% (w/v) to about 2% (w/v).

10. The composition of claim 1 wherein the first mydriatic compound is phenylephrine and the first pain-relieving compound is lidocaine.

11. The composition of claim 1 wherein the first mydriatic compound is tropicamide.

12. The composition of claim 11 wherein the tropicamide has a concentration from about 0.1% (w/v) to about 0.5% (w/v).

13. The composition of claim 1 further comprising a second mydriatic compound and a second pain-relieving compound.

14. The composition of claim 13, wherein the second mydriatic compound comprises epinephrine, phenylephrine, tropicamide, atropine, brimonidine, cyclopentolate, homatropine, 4-hydroxyamphetamine, or scopolamine, or pharmaceutically acceptable salts thereof.

15. The composition of claim 13, wherein the second pain-relieving compound comprises lidocaine, proparacaine, tetracaine, dexamethasone, fluorometholone, fluocinolone, loteprednol, loteprednol, difluprednate, triamcinolone, prednisolone, medrysone, or rimexolone, or pharmaceutically acceptable salts thereof.

16. The composition of claim 13 wherein the second pain-relieving compound is a non-steroidal anti-inflammatory drug.

17. The composition of claim 13 wherein the first mydriatic compound is tropicamide, the second mydriatic compound is phenylephrine, the first pain-relieving compound is lidocaine, and the second pain-relieving compound is diclofenac.

18. The composition of claim 17 wherein the tropicamide has a concentration from about 0.1% (w/v) to about 0.5% (w/v).

19. The composition of claim 17 wherein the phenylephrine has a concentration from about 0.01% (w/v) to about 2% (w/v).

20. The composition of claim 17 wherein the lidocaine has a concentration from about 0.5% (w/v) to about 2% (w/v).

21. The composition of claim 17 wherein the diclofenac has a concentration from about 0.01% (w/v) to about 0.5% (w/v).

22. The composition of claim 16 wherein the concentration of the non-steroidal anti-inflammatory drug is from about 0.1% (w/v) to about 0.5% (w/v).

23. The composition of claim 1 wherein the dye has a concentration from about 0.01% (w/v) to about 0.25% (w/v).

24. The composition of claim 1, wherein the composition is essentially free of preservatives and sulfites.

25. A method of delivering an ophthalmic pharmaceutical composition, the method comprising administering to an eye of a subject an ophthalmic pharmaceutical composition claim 1.
